(19) United States
(12) Patent Application Publication
(10) Pub. No.: US 2019/0183458 A1
(43) Pub. Date: Jun. 20, 2019

(54) PHOTOACOUSTIC GUIDE SUPPORT SYSTEM AND PHOTOACOUSTIC GUIDE SUPPORT METHOD

(71) Applicant: HITACHI, LTD., Tokyo (JP)

(72) Inventors: Ryo IMAI, Tokyo (JP); Tomohiko TANAKA, Tokyo (JP)

(73) Assignee: HITACHI, LTD., Tokyo (JP)

(21) Appl. No.: 16/219,509

(22) Filed: Dec. 13, 2018

(30) Foreign Application Priority Data
Dec. 15, 2017 (JP) 2017-241155

(51) Int. Cl. A61B 8/00; A61B 1/313; A61B 8/12; A61M 25/01; A61M 25/02

(52) U.S. Cl. CPC A61B 8/445; A61B 1/3137; A61M 2025/0037; A61M 25/0125; A61M 25/02; A61B 8/12

(57) ABSTRACT

According to one embodiment, a photoacoustic guide support system includes: a wire portion that is used in a catheter and includes an optical fiber provided therein and emitting a measuring laser beam into a blood vessel and a photoacoustic wave detection element which detects a photoacoustic wave generated when a target is irradiated with the measuring laser beam; and a signal processing device that determines whether the wire portion is capable of advancing based on a detection signal obtained when the photoacoustic wave detection element detects the photoacoustic wave. When the detection signal shows a predetermined pattern, the signal processing device determines that the wire portion is capable of advancing forward and displays the determination result to a display device.

### BACKGROUND OF THE INVENTION

### 1. Field of the Invention

The present invention relates to a technique of a photoacoustic guide support system and a photoacoustic guide support method used for catheterization.

### 2. Description of Related Art

As a treatment method for reestablishing blood flow of a blood vessel occluded by thrombus, there is a method of reestablishing blood flow using a small-diameter device such as a catheter. In this method, a catheter inserted from a wrist or an inguinal region is guided into an affected area to perform an examination or treatment of an occluded area. In this examination or treatment using a catheter, first, a wire having a diameter of several tenths of millimeters called a guide wire is inserted. Next, a catheter for examination or a catheter for treatment is introduced along the guide wire.

However, there is a case where it is difficult for the guide wire to penetrate some of lesions in which a blood vessel is occluded. A representative example is chronic total occlusion (CTO) of a coronary artery. The chronic total occlusion is a lesion in which the coronary artery is occluded for a long period of time of three months or longer. Since the blood vessel is completely occluded, blood stream cannot be observed by coronary arteriography with X-ray, and an upstream surface of the lesion may be hardened by calcification. In order to avoid penetration of the hardened area, a method called retrograde approach is frequently used.

In order to compensate for a decrease in blood flow rate caused by occlusion in CTO, a blood vessel called collateral channel is formed from another coronary artery. In the retrograde approach, the guide wire passes through the collateral channel such that the guide wire is penetrated from a far side of an occluded area where calcification has not occurred. However, unlike a typical coronary artery, the collateral channel has a small diameter, and the route thereof is meandering. Therefore, the penetration of the guide wire is more difficult than in a typical coronary artery. In consideration of the above-described circumstances, the degree of difficulty of a treatment for CTO is high, and a long-hour surgery is required for the treatment. The long-hour surgery is a large burden on a patient and a doctor, and it is necessary to intermittently image the heart using X-ray projection during catheterization. Therefore, the burden on the patient also increases due to an X-ray contrast agent or radiation exposure.

In order to solve the problem, intravascular information is obtained using a forward-viewing catheter or a guide wire in the treatment for CTO. It is considered to support the penetration of an occluded area using this method. In the catheterization, a vascular wall of a catheter side surface is observed by photoacoustic (ultrasonic) imaging or optical coherence tomography (OCT). If information regarding the forward side of a catheter can be obtained using this method, it is expected that a guide of the CTO treatment can be obtained.

For example, JP-T-10-506807 discloses a forward-viewing ultrasonic imaging catheter having a configuration in which “a simple forward-viewing ultrasonic catheter includes one or more transducers and ultrasonic mirrors, in which the transducers and the ultrasonic mirrors are supported by a bearing in a sealed end of a catheter, and a driving cable transmits a relative motion to the transducers and the mirror. The mirror leads the ultrasonic waves to the front of the catheter. An optical fiber can be provided to direct a laser beam for ablation of atheroma under the simultaneous intravascular ultrasonic guidance.” (see ABSTRACT).

In addition, WO2015/052852 discloses a blood vessel catheter system and a penetration method of a CTO lesion having a configuration in which “provided is a catheter system in which either an optical fiber catheter or a guide wire can be inserted into one lumen. With reference to an image that the optical fiber catheter is inserted into the lumen, the catheter (or the guide wire) is inserted into a CTO lesion. Next, the guide wire is inserted into the lumen to penetrate the CTO lesion.” (refer to ABSTRACT).

Here, there is a technical problem in that, for example, it is necessary to provide an acoustic wave receiving element on a front surface of a catheter or a guide wire, which has a diameter of about 1 mm or less, in order to perform forward imaging using photoacoustic waves (ultrasonic waves). On the other hand, in the case of OCT or intravascular endoscopy, blood needs to be removed using transparent liquid in order to prevent image deterioration caused by light scattering of the blood during imaging. Due to this problem, a forward-viewing device has not been widely used in the current CTO treatment.

On the other hand, recently, photoacoustic imaging has attracted attention as anew intravascular imaging method. In the photoacoustic imaging, a biological body is imaged by measuring a photoacoustic wave generated when the biological body is irradiated with a pulsed laser beam. In the photoacoustic imaging, the contrast in the optical absorption of a biological body can be imaged unlike a method of irradiating a biological body with ultrasonic waves. In addition, the photoacoustic imaging also has a characteristic in that there is little effect of light scattering of a biological body as compared to other optical imaging methods such as OCT. For example, Bo Wang et al., “Intravascular photoacoustic imaging of lipid in atherosclerotic plaques in the presence of luminal blood”, OPTIC LETTERS, Apr. 1, 2012, Vol. 37, No. 7, p. 1244 suggests a catheter for examination in which photoacoustic imaging is adopted.

As described above, in order to support the penetration of an occluded area, it is necessary to obtain information regarding the forward side of a catheter or the forward side of a guide wire. Further, the diameter of a catheter or a guide wire is required to be about 1 mm or less such that a device having the above-described mechanism can be inserted into a coronary artery. Further, the removal of a blood required in OCT or the like requires time and efforts, and thus it is desirable not to perform the removal.

In addition, when an occluded area in front is hardened by calcification or the like, it is not preferable that a force is applied to the guide wire for the penetration of the hardened area.

### SUMMARY OF THE INVENTION

The present invention has been made under the above-described circumstances, and an object thereof is to efficiently perform catheterization.

In order to solve the problem, according to the invention, a photoacoustic guide support system includes: a measuring laser beam generator that generates a measuring laser beam; a guide wire that includes an optical fiber for emitting the measuring laser beam to a target, and a detection portion for detecting a photoacoustic wave generated when the target is irradiated with the emitted measuring laser beam; and a signal processing portion that determines whether or not the guide wire is capable of advancing based on a detection signal obtained when the detection portion detects the photoacoustic wave, wherein when the detection signal shows a predetermined pattern, the signal processing portion determines that the guide wire is capable of advancing forward and outputs the determination result to an output portion.

Other solving means will be appropriately described in embodiments.

According to the present invention, catheterization can be efficiently performed.

### BRIEF DESCRIPTION OF THE DRAWINGS

FIG. 1A is a conceptual diagram illustrating a state where a photoacoustic guide wire used in an embodiment penetrates an occluded area;

FIG. 1B is an enlarged schematic cross-sectional view illustrating a part of the photoacoustic guide wire;

FIG. 2 is a diagram illustrating a configuration of a photoacoustic guide support system according to a first embodiment;

FIG. 3 is a diagram illustrating a hardware configuration of a signal processing device used in the first embodiment;

FIG. 4 is a flowchart illustrating a procedure of the photoacoustic guide support system that is performed in the first embodiment;

FIG. 5A is a diagram illustrating an example of a detection signal before correction;

FIG. 5B is a diagram illustrating an example of a correction function.

FIG. 5C is a diagram illustrating an example of a detection signal after correction;

FIG. 6 is a diagram illustrating a state where the photoacoustic guide wire reaches the occluded area;

FIG. 7A is a diagram (first) illustrating an example of a distance change of the detection signal depending on an absorption spectrum of the occluded area;

FIG. 7B is a diagram (second) illustrating an example of a distance change of the detection signal depending on an absorption spectrum of the occluded area;

FIG. 8A is a diagram (first) illustrating an example of a distance change of the detection signal depending on an absorption spectrum of blood;

FIG. 8B is a diagram (second) illustrating an example of a distance change of the detection signal depending on an absorption spectrum of the blood;

FIG. 9 is a diagram illustrating the photoacoustic guide wire in a blood vessel in which the occluded area is curved;

FIG. 10 is a diagram illustrating a configuration of a photoacoustic guide support system according to a second embodiment;

FIG. 11 is a diagram illustrating a hardware configuration of a signal processing device used in the second embodiment;

FIG. 12 is a diagram explaining a function of a photoacoustic guide wire used in the second embodiment;

FIG. 13 is a flowchart illustrating a procedure of the photoacoustic guide support system that is performed in the second embodiment;

FIG. 14 is a diagram (absorption spectrum of the occluded area) illustrating a detection signal obtained while the photoacoustic guide wire is rotating;

FIG. 15 is a diagram (absorption spectrum of the blood) illustrating a distance change of the detection signal obtained while the photoacoustic guide wire is rotating; and

FIG. 16 is a schematic diagram illustrating a system structure of a photoacoustic guide support system according to a third embodiment.

### DESCRIPTION OF EMBODIMENTS

Next, embodiments for implementing the present invention (referred to as “embodiment”) will be described in detail appropriately with reference to the drawings.

### Conceptual Diagram

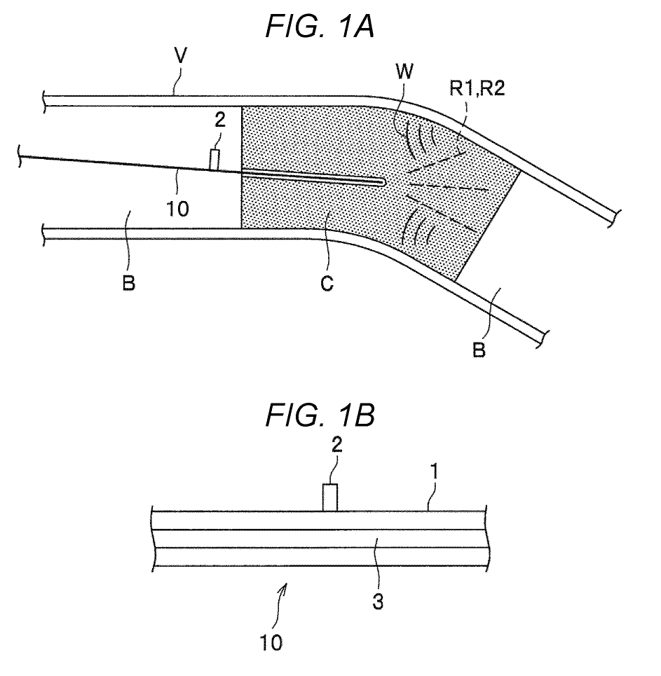

FIG. 1A is a conceptual diagram illustrating a state where a photoacoustic guide wire 10 used in an embodiment penetrates an occluded area C. In addition, FIG. 1B is an enlarged schematic cross-sectional view illustrating a part of the photoacoustic guide wire 10.

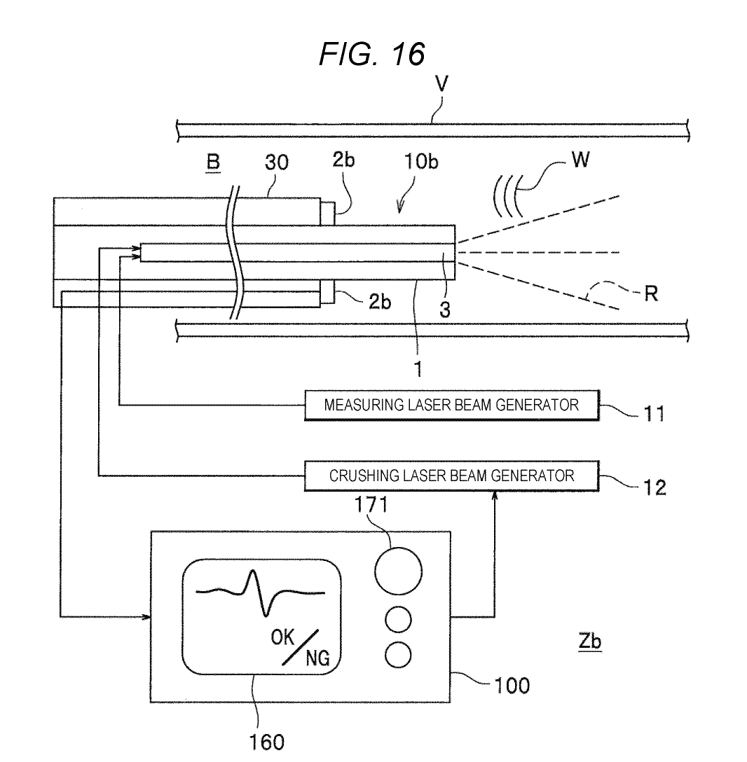

Here, the photoacoustic guide wire 10 is used in a catheter, but the catheter is not illustrated in the drawings other than FIG. 16.

As illustrated in FIG. 1B, in the photoacoustic guide wire 10 according to the embodiment, an optical fiber 3 is provided in a wire portion (guide wire) 1. Further, the photoacoustic guide wire 10 is characterized in that a photoacoustic wave detection element (detection portion) 2 that detects a photoacoustic wave W is provided.

As illustrated in FIG. 1A, the photoacoustic guide wire 10 is inserted into a blood vessel V and advances through blood B that is filled in the blood vessel V.

In the embodiment, as illustrated in FIG. 1A, a pulsed laser beam for measurement (measuring laser beam R1) is emitted from a tip of the optical fiber 3 to the front of the photoacoustic guide wire 10. A photoacoustic wave W generated when a biological body is irradiated with the measuring laser beam R1 is detected by the photoacoustic wave detection element 2. A state of a forward tissue is determined based on signal intensity and a waveform of a detection signal generated from the photoacoustic wave detection element 2 when the photoacoustic wave W is detected by the photoacoustic wave detection element 2.

When it is determined that the photoacoustic guide wire 10 can advance based on the result of the determination on the state of the tissue, a doctor advances the photoacoustic guide wire 10. However, when the forward occluded area C is hardened by calcification or the like, it may be difficult to advance the photoacoustic guide wire 10. In this case, a laser beam for crushing a forward tissue (crushing laser beam R2) is emitted from the optical fiber 3. As a result, the occluded area C such as thrombus is crushed such that the advancement of the photoacoustic guide wire 10 is assisted.

Hereinafter, a specific configuration or a specific operation of the photoacoustic guide wire 10 illustrated in FIGS. 1A and 1B will be described.

### First Embodiment

### System

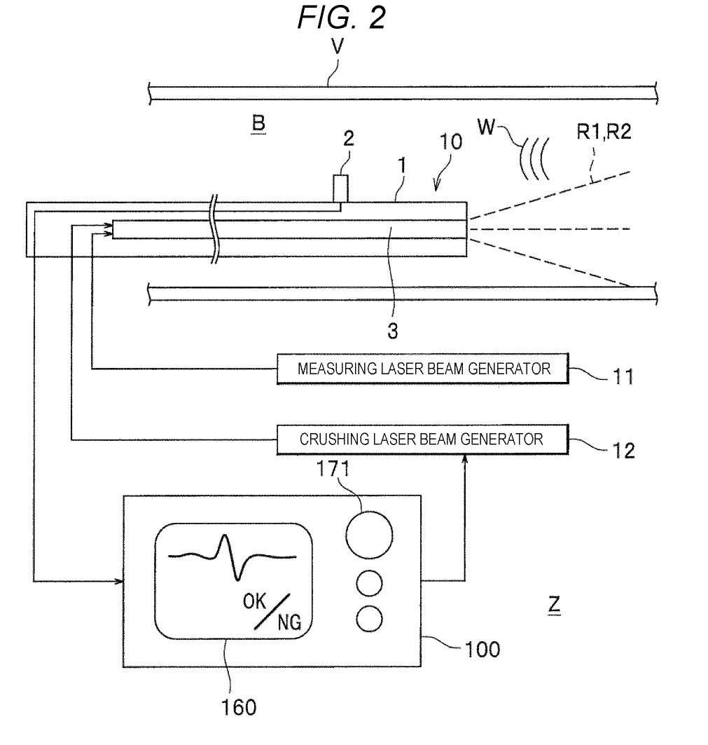

FIG. 2 is a diagram illustrating a configuration of a photoacoustic guide support system Z according to a first embodiment. In FIG. 2, the same components as those of FIG. 1 are denoted by the same reference numerals, and the description thereof will not be repeated. In order not to make the drawings complicated, the occluded area C is not illustrated in FIG. 2. In addition, in FIG. 2, for easy understanding of the drawing, the size of the photoacoustic guide wire 10 with respect to the blood vessel V is illustrated to be larger than the actual size. The same shall be applied to other drawings.

In the photoacoustic guide support system Z, the photoacoustic guide wire 10 includes the optical fiber 3 provided in the wire portion 1 that is the same as a guide wire used for catheterization and the photoacoustic wave detection element 2. As illustrated in FIG. 2, the photoacoustic wave detection element 2 is provided on a side surface of the wire portion 1. The optical fiber 3 is connected to a measuring laser beam generator 11 that is connected to a base of the wire portion 1. The measuring laser beam R1 that is incident from the measuring laser beam generator 11 to the optical fiber 3 is emitted from the tip portion of the optical fiber 3. The photoacoustic wave W, which is an ultrasonic wave generated when a biological body is irradiated with the measuring laser beam R1, is detected by the photoacoustic wave detection element 2. The photoacoustic wave detection element 2 converts an intensity of the photoacoustic wave W into an electric signal (detection signal) and transmits this detection signal to a signal processing device (signal processing portion) 100. The signal processing device 100 determines an advancing direction, that is, whether or not the occluded area C can be crushed by the laser beam, based on the transmitted detection signal. The determination on the advancing direction will be described below. The result (OK/NG) of the determination of the signal processing device 100 on the advancing direction is displayed on a display device (output portion) 160. When a crushing laser beam emission button 171 is pressed toward the determined advancing direction, an instruction signal is transmitted to a crushing laser beam generator 12. The crushing laser beam R2 generated by the crushing laser beam generator 12 is emitted from the tip portion of the optical fiber 3. The power of the crushing laser beam R2 is stronger than that of the measuring laser beam R1. The emitted crushing laser beam R2 crushes a tissue in the vicinity of the tip portion of the photoacoustic guide wire 10.

### Signal Processing Device 100

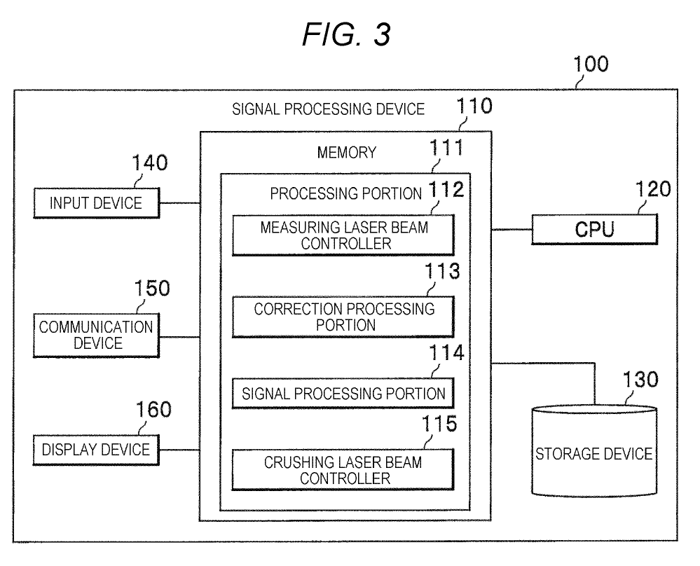

FIG. 3 is a diagram illustrating a hardware configuration of the signal processing device 100 used in the first embodiment. The description will be made appropriately with reference to FIG. 2.

The signal processing device 100 includes a memory 110, a central processing unit (CPU) 120, and a storage device 130 such as a hard disk (HD). Further, the signal processing device 100 includes an input device 140, a communication device 150, and the display device 160.

On the memory 110, a program stored in the storage device 130 is loaded. The loaded program is executed by the CPU 120. As a result, a processing portion 111, and a measuring laser beam controller 112, a correction processing portion 113, a signal processing portion 114 and a crushing laser beam controller 115 that constitute the processing portion 111 are implemented.

The measuring laser beam controller 112 causes the measuring laser beam generator 11 to generate the measuring laser beam R1.

The correction processing portion 113 corrects a signal intensity using a method described below.

The signal processing portion 114 determines a direction in which the photoacoustic guide wire 10 is to advance using a method described below.

When the crushing laser beam emission button 171 (refer to FIG. 2) is pressed, the crushing laser beam controller 115 causes the crushing laser beam generator 12 to generate the crushing laser beam R2.

The input device 140 includes the crushing laser beam emission button 171 illustrated in FIG. 2.

The communication device 150 receives the detection signal from the photoacoustic wave detection element 2. In addition, the communication device 150 transmits an instruction signal to the measuring laser beam generator 11 to generate the measuring laser beam R1, or transmits an instruction signal to the crushing laser beam generator 12 to generate the crushing laser beam R2.

The display device 160 has been described above with reference to FIG. 2, and thus the description thereof will not be repeated here.

### Procedure

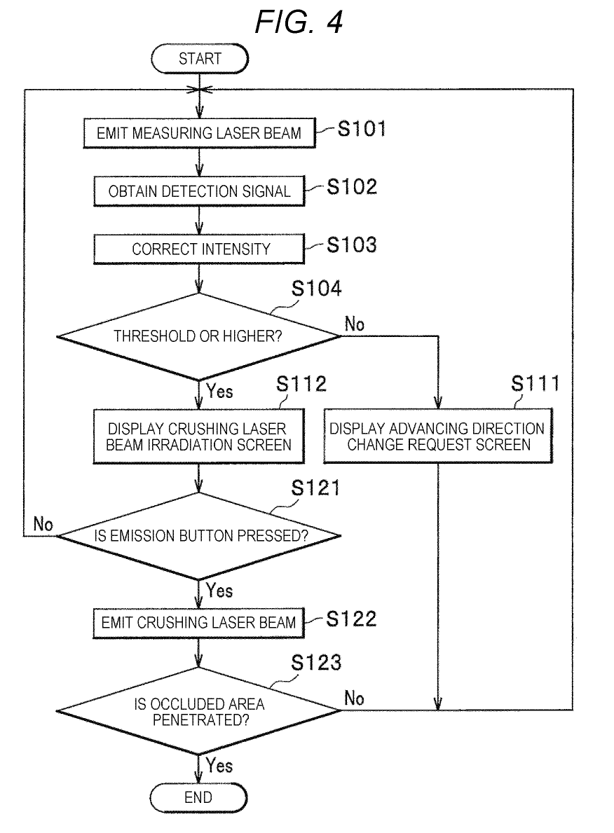

FIG. 4 is a flowchart illustrating a procedure of the photoacoustic guide support system Z that is performed in the first embodiment.

First, the process of FIG. 4 starts after the tip portion of the photoacoustic guide wire 10 is inserted by the doctor up to a position where the tip portion of the photoacoustic guide wire 10 cannot advance easily. It is not necessary to strictly determine whether or not the position of the tip portion of the photoacoustic guide wire 10 is the position where the tip portion of the photoacoustic guide wire 10 cannot advance easily. For example, when the doctor determines that the tip portion of the photoacoustic guide wire 10 reaches a coronary artery, the process of FIG. 4 may be performed.

After the start of the operation, the measuring laser beam controller 112 causes the measuring laser beam generator 11 to generate the measuring laser beam R1. As a result, the measuring laser beam R1 is emitted from the tip of the optical fiber 3 (S101).

When a target is irradiated with the measuring laser beam R1, the photoacoustic wave W is generated from the target.

Next, the detection signal of the photoacoustic wave W is detected by the photoacoustic wave detection element 2 (S102).

Next, the correction processing portion 113 corrects an influence of light diffusion and absorption (intensity correction) (S103). The process of Step S103 will be described below.

Next, the signal processing portion 114 determines whether or not a specific peak value in the detection signal is a threshold or higher (predetermined pattern) (S104). The determination depending on whether the specific peak value is the threshold or higher will be described below.

When the specific peak value in the detection signal is lower than the threshold as a result of Step S104 (S104→No), the signal processing portion 114 displays an advancing direction change request screen on the display device 160 (S111). On the advancing direction change request screen, information indicating that it is necessary to change the advancing direction is displayed. Next, the processing portion 111 returns to the process of Step S101.

When the specific peak value in the detection signal is the threshold or higher as a result of Step S104 (S104→4 Yes), the signal processing portion 114 displays a crushing laser beam irradiation screen on the display device 160 (S112). The crushing laser beam irradiation screen displays information indicating that the crushing laser beam R2 can be irradiated in a direction where the photoacoustic guide wire 10 is currently facing.

Next, the crushing laser beam controller 115 determines whether or not the crushing laser beam emission button 171 (emission button) is pressed by the doctor (S121).

When the crushing laser beam emission button 171 is not pressed by the doctor (S121→No) as a result of Step S121, the processing portion 111 returns to the process of Step S101.

When the crushing laser beam emission button 171 is pressed by the doctor as a result of Step S121 (S121→Yes), the crushing laser beam controller 115 causes the crushing laser beam generator 12 to generate the crushing laser beam R2. As a result, the crushing laser beam R2 is emitted from the tip of the optical fiber 3 (S122).

Next, the measuring laser beam controller 112 causes the measuring laser beam R1 to be emitted from the tip of the optical fiber 3. After the processing portion 111 performs the same processes as those of Steps S102 and S103, the signal processing portion 114 determines whether or not the occluded area C is penetrated based on the detection signal (S123).

When the occluded area C is not penetrated as a result of Step S123 (S123→No), the processing portion 111 returns to the process of Step S101.

When the occluded area C is penetrated as a result of Step S123 (S123→Yes), the processing portion 111 ends the process.

When the tip portion of the photoacoustic guide wire 10 is inserted again by the doctor up to the position where the tip portion of the photoacoustic guide wire 10 cannot advance easily, the photoacoustic guide support system Z performs the processes from Step S101 again.

### Intensity Correction Process: S103 of FIG. 4

When the photoacoustic wave W is detected by the photoacoustic wave detection element 2, a time-series detection signal is generated. The elapsed time from when the measuring laser beam R1 is emitted corresponds to the distance from the tip portion of the photoacoustic guide wire 10. The distance L from the tip portion of the photoacoustic guide wire 10 to the target that is irradiated with the measuring laser beam R1 is represented by the following Expression (1).

L=(Δt×v−L₀)/2 (1)

Here, Δt represents the elapsed time from when the measuring laser beam R1 is emitted from the tip of the optical fiber 3. In addition, v represents a sound velocity in the body, and L₀ represents the distance from the tip of the photoacoustic guide wire 10 to the photoacoustic wave detection element 2. After the emission from the optical fiber 3, the measuring laser beam R1 is diffused and absorbed such that the signal intensity thereof decreases. Therefore, it is necessary to correct the detection signal using a correction function with respect to Δt, that is, the distance from the tip of the optical fiber 3.

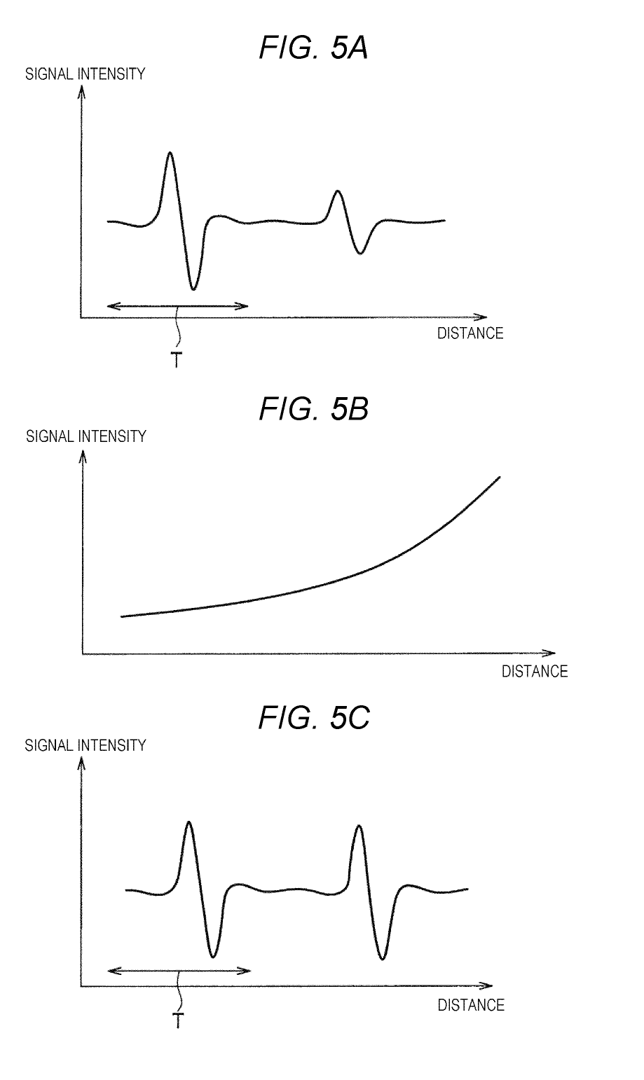

That is, as illustrated in FIG. 5B, the correction processing portion 113 corrects the detection signal according to a correction curve of the detection signal with respect to the distance from the tip of the optical fiber 3.

FIGS. 5A to 5C are diagrams illustrating examples of the intensity correction performed in Step S103 of FIG. 4.

Here, FIG. 5A illustrates an example of the detection signal before correction, FIG. 5B illustrates an example of the correction function, and FIG. 5C illustrate an example of the detection signal after correction.

In FIGS. 5A to 5C, the horizontal axis represents the distance from the tip of the optical fiber 3. That is, the origin of the horizontal axis represents the tip of the optical fiber 3. In addition, the vertical axis represents the signal intensity. In FIGS. 5A and 5C, the symbol T represents the detection signal transmitted from the near side of the occluded area C.

As illustrated in FIG. 5A, the signal intensity of the obtained detection signal becomes weak as the distance increases.

Therefore, the correction processing portion 113 amplifies the signal intensity of the signal obtained from a long distance using the correction curve illustrated in FIG. 5B. AS illustrated in FIG. 5B, the correction curve is set such that the signal intensity is amplified as the distance increases.

When the intensity correction using the correction function is performed on the detection signal illustrated FIG. 5A, even the signal obtained from a long distance is restored to the detection signal having the original signal intensity as illustrated in FIG. 5C.

### Determination on Advancing Direction: S104 of FIG. 4

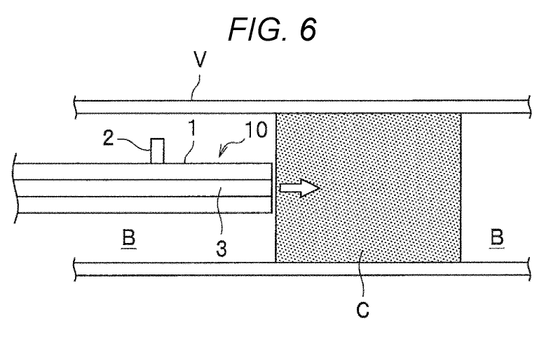

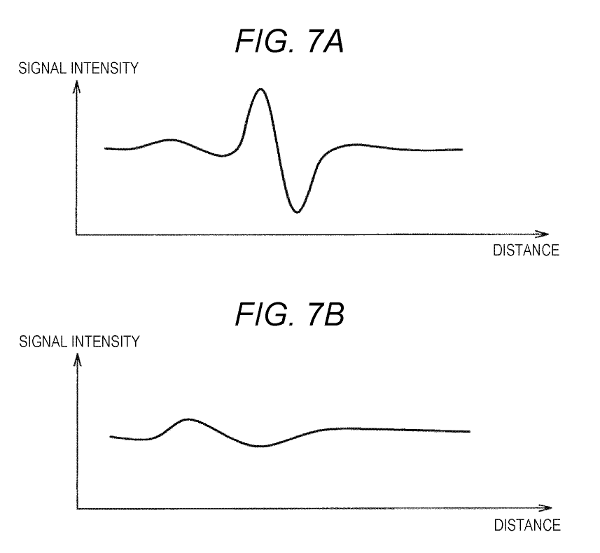

A method of performing the determination on whether or not the photoacoustic guide wire 10 can advance and the determination on the advancing direction will be described with reference to FIGS. 6, 7A and 7B. This process corresponds to Step S104 of FIG. 4. In FIG. 6, the same components as those of FIG. 1 are denoted by the same reference numerals, and the description thereof will not be repeated. In addition, the horizontal axis and the vertical axis in FIGS. 7A and 7B are the same as those in FIGS. 5A and 5C. Further, the detection signals illustrated in FIGS. 7A and 7B have undergone the intensity correction.

First, it is assumed that the process of the determination on the advancing direction according to the embodiment is performed only when it is difficult to advance the photoacoustic guide wire 10 due to a hardened area of the occluded area C in the blood vessel V. That is, as illustrated in FIG. 6, when the photoacoustic guide wire 10 reaches the occluded area C and is not likely to further advance, first, the measuring laser beam R1 is emitted. In FIG. 6, a white arrow indicates an emission direction of the measuring laser beam R1. In photoacoustic imaging, it is known that, when a tissue is irradiated with light (laser beam) having a given wavelength, the photoacoustic wave W corresponding to the intensity of absorbed light is generated from a specific tissue that absorbs the light (laser beam) having the wavelength. For example, it is known that the blood B strongly absorbs light having a wavelength of about 600 nm or shorter, and fat strongly absorbs light having a wavelength of about 1200 nm or 1700 nm.

FIGS. 7A and 7B are diagrams illustrating examples of a distance change of the detection signal when the wavelength of the measuring laser beam R1 is matched to an absorption spectrum of a tissue of the occluded area C. Incidentally, when the wavelength of the measuring laser beam R1 is matched to the absorption spectrum of the tissue of the occluded area C, the measuring laser beam R1 is absorbed by the surface of the occluded area C. Therefore, the measuring laser beam R1 does not penetrate the inside of the occluded area C.

In the photoacoustic guide wire 10 according to the embodiment, it is desirable that the wavelength of the measuring laser beam R1 matches with a characteristic absorption wavelength in the tissue of the occluded area C. In this case, when the occluded area C is positioned in front of the photoacoustic guide wire 10, the photoacoustic wave W having a strong peak as illustrated in FIG. 7A is generated. That is, the peak of the detection signal in FIG. 7A is derived from the photoacoustic wave W emitted from the occluded area C.

In addition, when vascular wall (or the blood B) is positioned in front of the photoacoustic guide wire 10, the photoacoustic wave W having a weak peak as illustrated in FIG. 7B is generated.

That is, when the peak value of the detection signal after the intensity correction in Step S103 of FIG. 4 is higher than the predetermined threshold, the signal processing portion 114 may determine that the occluded area C is positioned in front of the photoacoustic guide wire 10. When the occluded area C is positioned in front of the photoacoustic guide wire 10, the emission of the crushing laser beam R2 is performed. More specifically, when the peak value of the detection signal present at a predetermined distance or longer from the tip of the optical fiber 3 is higher than the predetermined threshold, the signal processing portion 114 determines that the direction in which the photoacoustic guide wire 10 faces is the advancing direction.

In this case, for example, the doctor rotates the photoacoustic guide wire 10 in various ways such that the signal processing portion 114 determines a direction in which the peak value illustrated in FIG. 7A is the highest as the direction in which the photoacoustic guide wire 10 is to advance.

A case where the detection signal after the intensity correction is higher than the predetermined threshold as illustrated in FIG. 7A corresponds to the case where the specific peak value is the threshold or higher in Step S104 of FIG. 4.

As such, by using the measuring laser beam R1 having a wavelength in the absorption spectrum of the tissue of occluded area C, a direction in which the occluded area C is present, that is, the advancing direction of the photoacoustic guide wire 10 is determined.

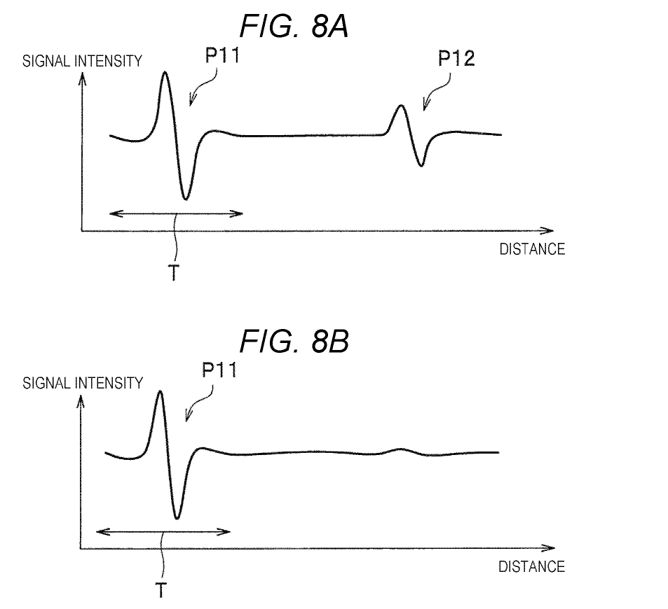

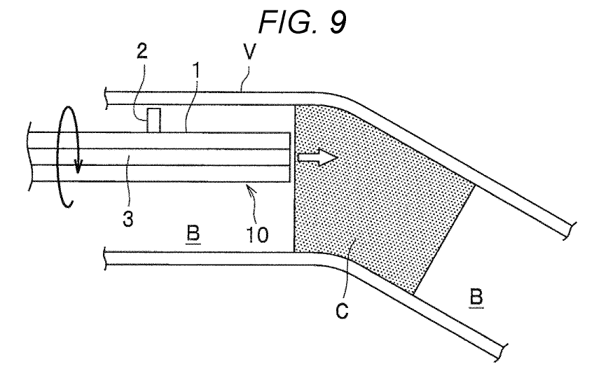

FIGS. 8A and 8B are diagrams illustrating examples of a distance change of the detection signal when the wavelength of the measuring laser beam R1 is matched to an absorption spectrum of the blood B. The horizontal axis and the vertical axis in FIGS. 8A and 8B are the same as those in FIGS. 5A and 5C. Further, the detection signals illustrated in FIGS. 8A and 8B have undergone the intensity correction.

As illustrated in FIGS. 8A and 8B, the wavelength of the measuring laser beam R1 may be matched to the absorption spectrum of a biological body, in particular, the blood B having strong light absorption.

In this case, the detection signal derived from the blood B remaining in the occluded area C or the blood B positioned behind the occluded area C can be obtained. Even in this case, when the detection signal is a predetermined value or higher, crushing is performed.

Here, FIG. 8A is a diagram illustrating a distance change of the detection signal when the occluded area C is present in front of the photoacoustic guide wire 10 and the blood B is further present in front of the occluded area C. That is, FIG. 8A is a diagram illustrating a distance change of the detection signal obtained from the state illustrated in FIG. 6.

In FIG. 8A, a peak P11 is a reaction that is derived from the blood B present on the near side of the occluded area C, and a peak P12 is a reaction that is derived from the blood B on the far side of the occluded area C. In the embodiment, the near side refers to the near side when seen from the photoacoustic guide wire 10, and the far side refers to the far side when seen from the photoacoustic guide wire 10.

On the other hand, FIG. 8B is a diagram illustrating a distance change of the detection signal when the vascular wall is present on the far side of the occluded area C.

That is, FIG. 8B is a diagram illustrating a distance change of the detection signal when the blood vessel of the occluded area C is curved (refer to FIG. 9). In FIG. 9, the same components as those of FIGS. 1 and 2 are denoted by the same reference numerals, and the description thereof will not be repeated. In addition, in FIG. 9, a white arrow indicates an emission direction of the measuring laser beam R1. In FIGS. 8A and 8B, the symbol T represents the detection signal transmitted from the near side of the occluded area C.

In FIG. 8B, as in the case of FIG. 8A, a peak P11 is a reaction that is derived from the blood B present on the near side of the occluded area C. However, in this case, as illustrated in FIG. 9, the blood B is not present on the far side of the occluded area C in the emission direction of the measuring laser beam R1. Therefore, a peak corresponding to the peak P12 of FIG. 8A is not present.

In this case, the signal processing portion 114 determines a direction in which the pattern illustrated in FIG. 8A is obtained as the advancing direction of the photoacoustic guide wire 10. Further, the signal processing portion 114 determines a direction in which the peak P12 of FIG. 8A is the highest (the threshold or higher) as the advancing direction of the photoacoustic guide wire 10.

That is, the specific peak value in Step S104 of FIG. 4 refers to the value of the peak P12.

When the photoacoustic guide wire 10 does not still penetrate the inside of the occluded area C, it is necessary to distinguish the detection signal of the blood B present on the near side of the occluded area C (refer to FIG. 6). Basically, the embodiment is the case using the photoacoustic guide wire 10 when the photoacoustic guide wire 10 reaches a portion immediately before the occluded area C as illustrated in FIG. 6. Therefore, it is conceivable that the thickness of a layer of the blood B on the near side of the occluded area C is limited. Accordingly, the detection signal corresponding to a predetermined distance, for example, 1 mm from the tip portion of the photoacoustic guide wire 10 may be ignored. As a result, the risk of making an erroneous determination based on the detection signal obtained from the blood B on the near side of the occluded area C can be avoided. That is, by ignoring the peak 11 in FIG. 8A or 8B, the signal processing portion 114 can determine the advancing direction only based on the peak 12.

The measuring laser beam R1 having the absorption spectrum of the blood B may be used when whether or not the occluded area C is present near the photoacoustic guide wire 10 is not clear. As such, by using the measuring laser beam R1 having the absorption spectrum of the blood B, a guide of the advancing direction can be obtained even when whether or not the occluded area C is present near the photoacoustic guide wire 10 is not clear. In addition, by using the measuring laser beam R1 having the absorption spectrum of a biological body, in particular, the blood B having strong light absorption, the advancing direction can be determined with high accuracy.

The measuring laser beam R1 having the absorption spectrum of the blood B and the measuring laser beam R1 having the absorption spectrum of the tissue of the occluded area C can be selectively used according to the circumstances.

It is known that, regarding the photoacoustic wave W generated by the irradiation of the measuring laser beam R1, the signal intensity and the waveform vary depending on optical characteristics of the tissue of the irradiated portion. By recognizing the forward state of the photoacoustic guide wire 10 based on the information of the photoacoustic wave W, the advancement of the photoacoustic guide wire 10 in a direction that is not the advancing direction can be avoided. In addition, even when the photoacoustic guide wire 10 enters a false lumen, a guide for returning the photoacoustic guide wire 10 to the inside of the original blood vessel V (true lumen) can be obtained. Further, when a calcified portion is present in front of the photoacoustic guide wire 10, the calcified portion is crushed by the crushing laser beam R2 such that the photoacoustic guide wire 10 can easily advance.

In a hole that is formed in the occluded area C by the photoacoustic guide wire 10 according to the embodiment, for example, a balloon or a stent is provided. That is, by emitting the crushing laser beam R2 in a direction that is determined as the advancing direction, the hole for providing a balloon or a stent can be obtained.

In addition, since the photoacoustic wave detection element 2 is provided on the wire portion 1, the advancing direction can be determined based on the detection signal obtained by the photoacoustic guide wire 10 itself.

### Second Embodiment

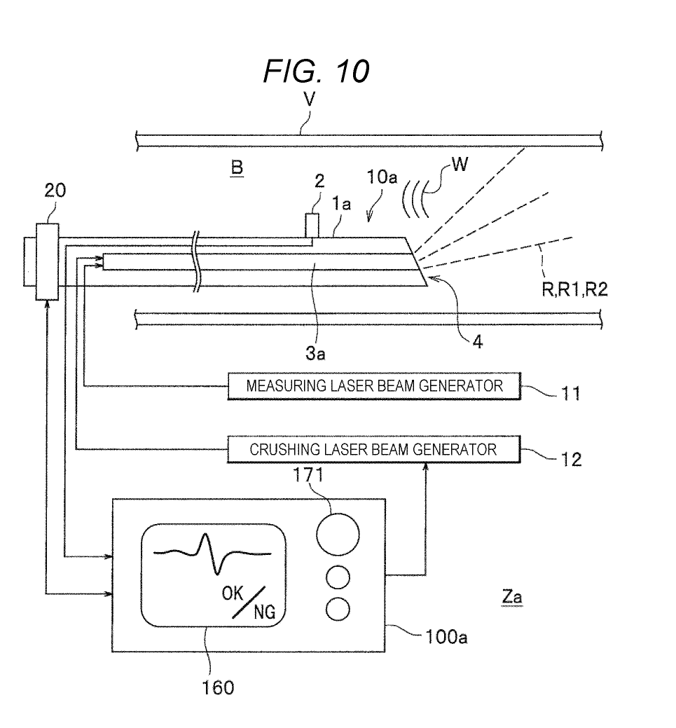

Next, a second embodiment of the present invention will be described with reference to FIGS. 10 to 15.

In the first embodiment, whether or not the photoacoustic guide wire 10 can advance forward is determined. On the other hand, in the second embodiment, the advancing direction is indicated.

### Photoacoustic Guide Support System Za

FIG. 10 is a diagram illustrating a configuration of a photoacoustic guide support system Za according to the second embodiment.

Differences between the signal processing device 100 illustrated in FIG. 2 according to the first embodiment and the photoacoustic guide support system Za illustrated in FIG. 10 are as follows.

(1) A photoacoustic guide wire 10a includes a refraction portion 4 that is configured such that a laser beam R is emitted in an oblique direction with respect to a central axis of an optical fiber 3a. Here, the laser beam R includes the measuring laser beam R1 and the crushing laser beam R2. The refraction portion 4 may have a configuration in which a tip of the optical fiber 3a (and a wire portion 1a) is cut obliquely as illustrated in FIG. 10, or may have a configuration in which the tip portion of the optical fiber 3 (refer to FIG. 2) is curved. Alternatively, as the refraction portion 4, a lens or the like may be used. Here, the tip of the optical fiber 3a (and the wire portion 1a) may be configured to be cut obliquely.

(2) A rotation control device (rotation controller, rotation angle detection portion) 20 that controls rotation of the photoacoustic guide wire 10a and measures a rotation angle of the photoacoustic guide wire 10a is provided in the photoacoustic guide wire 10a.

(3) A signal processing device 100a controls the rotation of the photoacoustic guide wire 10a.

Since the other configurations are the same as those of the photoacoustic guide support system Z illustrated in FIG. 2, the same components as those in FIG. 2 are denoted by the same reference numerals, and the description thereof will not be repeated.

### Signal Processing Device 100a

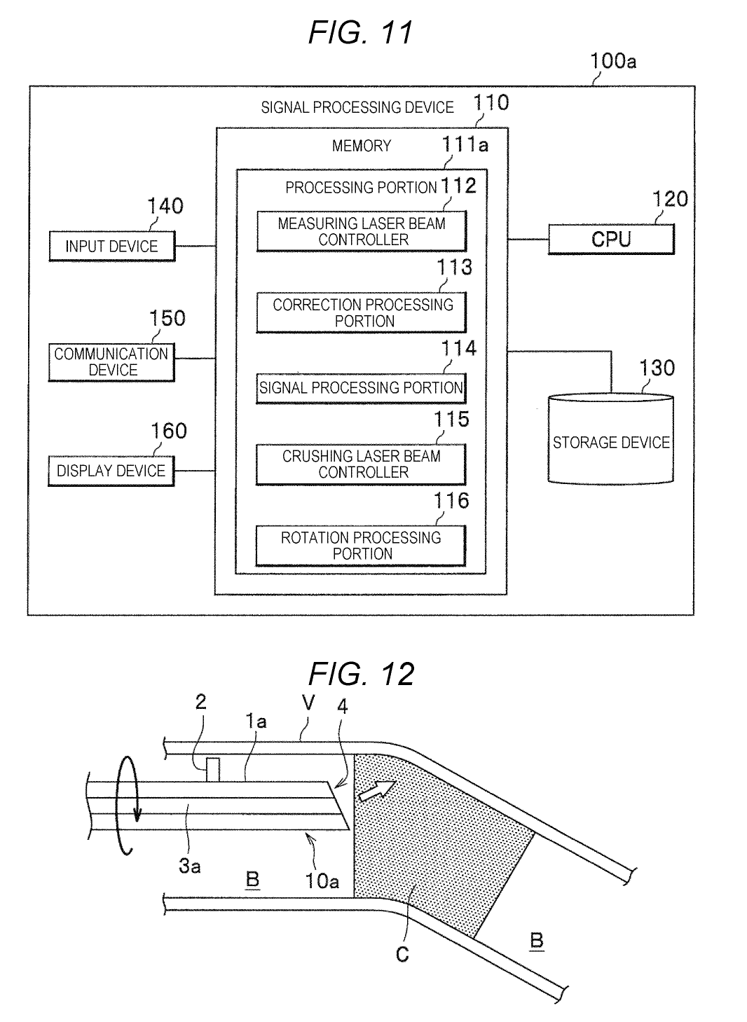

FIG. 11 is a diagram illustrating a hardware configuration of the signal processing device 100a used in the second embodiment. In FIG. 11, the same components as those of FIG. 3 are denoted by the same reference numerals, and the description thereof will not be repeated. The description will be made appropriately with reference to FIG. 10.

A difference of the signal processing device 100a from the signal processing device 100 illustrated in FIG. 3 is that a processing portion 111a includes a rotation processing portion (rotation controller) 116.

The rotation processing portion 116 causes the rotation control device 20 to rotate the photoacoustic guide wire 10a and obtains the rotation angle of the photoacoustic guide wire 10a transmitted from the rotation control device 20.

FIG. 12 is a diagram explaining a function of the photoacoustic guide wire 10a used in the second embodiment. In FIG. 12, the same components as those of FIG. 10 are denoted by the same reference numerals, and the description thereof will not be repeated.

In the second embodiment, as in the first embodiment, it is assumed that the photoacoustic guide wire 10a advances up to a position where the photoacoustic guide wire 10a cannot advance easily.

Here, an appropriate advancing direction is determined by rotating the photoacoustic guide wire 10a. It is desirable that the photoacoustic guide wire 10a is automatically rotated by the rotation control device 20. However, the photoacoustic guide wire 10a may be manually rotated. During the rotation of the photoacoustic guide wire 10a, the measuring laser beam R1 is emitted from the tip of the optical fiber 3a (indicated by a white arrow in FIG. 12). The emitted measuring laser beam R1 is emitted by the refraction portion 4 in an oblique direction with respect to the central axis of the optical fiber 3a. As a result, the measuring laser beam R1 is also emitted while being rotated.

Detection signals are detected from respective rotation angles. The detected detection signals are associated with each other by the signal processing device 100a. This configuration will be described below.

It is assumed that the blood vessel V, the occluded area C, the blood B, and the photoacoustic guide wire 10a have a positional relationship illustrated in FIG. 12. In addition, the wavelength of the emitted measuring laser beam R1 has a characteristic absorption wavelength with respect to the occluded area C as in the case of the first embodiment. In the case of the positional relationship illustrated in FIG. 12, a direction in which the photoacoustic guide wire 10a is to advance is a lower right direction. Here, in order to determine the direction in which the photoacoustic guide wire 10a is to advance, first, the photoacoustic wave detection element 2 detects the photoacoustic wave W while the photoacoustic guide wire 10a is rotating.

### Procedure

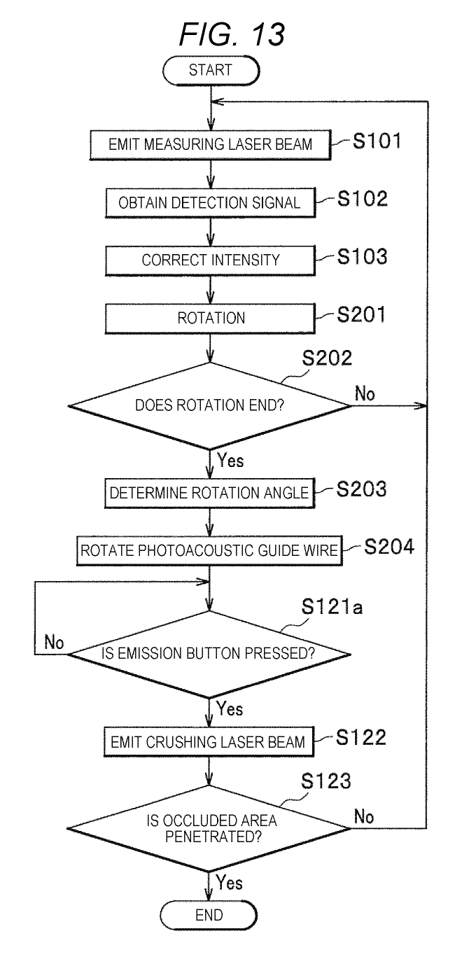

FIG. 13 is a flowchart illustrating a procedure of the photoacoustic guide support system. Za that is performed in the second embodiment. In FIG. 13, the same processes as those of FIG. 4 are denoted by the same step numbers, and the description thereof will not be repeated. The description will be made appropriately with reference to FIG. 11.

In Step S103, after the intensity correction, the rotation processing portion 116 rotates the photoacoustic guide wire 10a at a predetermined angle (S201). The processes of Steps S101 to S103 are processes of obtaining the detection signal corresponding to one rotation angle.

The rotation processing portion 116 determines whether or not the rotation ends (S202). That is, the rotation processing portion 116 determines whether or not the photoacoustic guide wire 10a is rotated by 360 degrees based on the rotation angle obtained from the rotation control device 20.

When the rotation does not end as a result of Step S202 (S202→No), the processing portion 111a returns to the process of Step S101.

When the rotation ends as a result of Step S202 (S202→Yes), the rotation processing portion 116 determines the rotation angle (S203). A method of determining the rotation angle will be described below. At this time, the determined rotation angle may be displayed on the display device 160.

The rotation processing portion 116 rotates the photoacoustic guide wire 10a to the rotation angle determined in Step S203 (S204).

Next, the crushing laser beam controller 115 determines whether or not the crushing laser beam emission button 171 (emission button) is pressed by the doctor (S121a).

When the crushing laser beam emission button 171 is not pressed by the doctor as a result of Step S121a (S121a→No), the processing portion 111a returns to the process of Step S121a.

When the crushing laser beam emission button 171 is pressed by the doctor as a result of Step S121a (S121a→Yes), the crushing laser beam controller 115 causes the crushing laser beam R2 to be emitted (S122). Then, the processing portion 111a performs the process of Step S123.

### Determination on Rotation Angle: S203 of FIG. 13

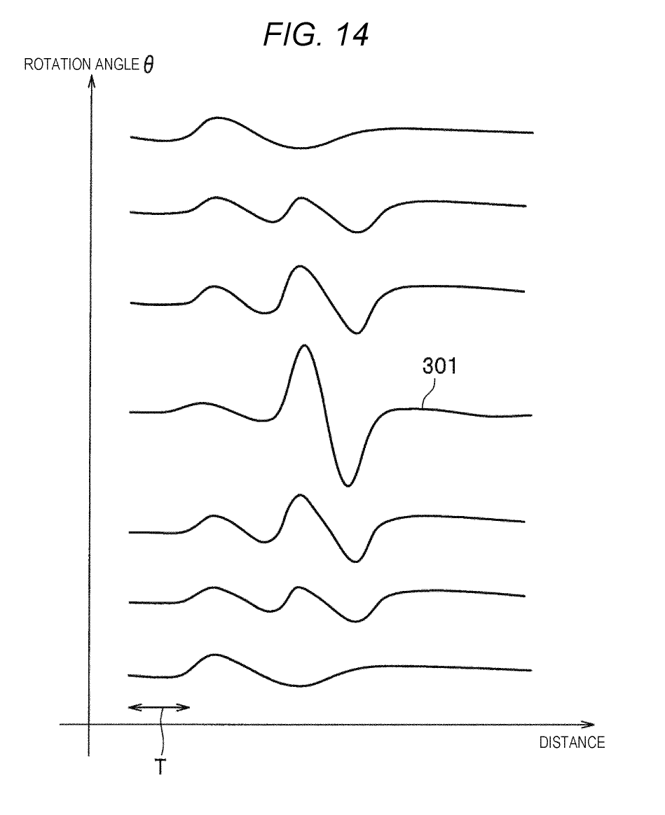

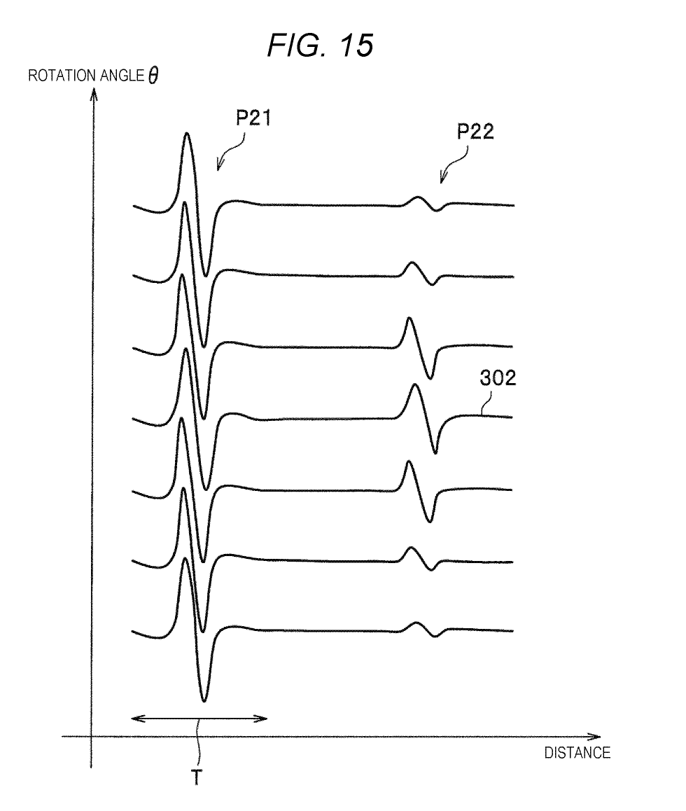

Here, the process (S203) of determining the rotation angle in FIG. 13 will be described with reference to FIGS. 14 and 15.

FIG. 14 is a diagram illustrating the detection signal obtained while the photoacoustic guide wire 10a is rotating in the example illustrated in FIG. 12. FIG. 14 illustrates an example in which the absorption spectrum of the measuring laser beam R1 is matched to the absorption spectrum of the tissue of the occluded area C. Therefore, in each of detection signals obtained in respective angles in the example of FIG. 14, only one peak appears.

In FIG. 14, the horizontal axis represents the distance from the tip of the optical fiber 3, and the vertical axis represents the rotation angle θ. In addition, the symbol T represents the detection signal transmitted from the near side of the occluded area C.

The detection signal becomes weak as the measuring laser beam faces in a direction of the vascular wall, and the detection signal becomes strong as the measuring laser beam faces in a direction of the occluded area C. Accordingly, a direction in which the peak of the detection signal is the highest is the advancing direction. In the example of FIG. 14, the peak of a detection signal 301 is the highest (predetermined pattern). Therefore, a direction of the rotation angle θ at which the detection signal 301 is obtained is the advancing direction.

As such, by using the measuring laser beam R1 having a wavelength in the absorption spectrum of the tissue of occluded area C, a direction in which the occluded area C is present, that is, the advancing direction of the photoacoustic guide wire 10 is determined.

FIG. 15 is a diagram illustrating a distance change of the detection signal obtained while the photoacoustic guide wire 10a is rotating in the example of FIG. 12. Here, the symbol T represents the detection signal transmitted from the near side of the occluded area C.

FIG. 15 illustrates an example in which the absorption spectrum of the measuring laser beam R1 is matched to the absorption spectrum of the tissue of the blood B. Therefore, in each of detection signals obtained in individual angles in the example of FIG. 14, two peaks P21 and P22 appear. That is, the peak P21 is derived from the blood B present on the near side of the occluded area C, and the peak P22 is derived from the blood B present on the far side of the occluded area C (refer to FIG. 8A).

In FIG. 15, as in the case of FIG. 14, the horizontal axis represents the distance from the tip of the optical fiber 3, and the vertical axis represents the rotation angle θ.

Referring to FIG. 12, the peak 22 in FIG. 15 is low when the measuring laser beam R1 faces a direction opposite to a direction in which blood vessel V is curved. In addition, referring to FIG. 12, the peak P22 in FIG. 15 is high when the measuring laser beam R1 faces a lower right direction. Accordingly, a direction in which the peak P22 is the highest (predetermined pattern) is the advancing direction. In the example of FIG. 15, the peak P22 of a detection signal 302 is the highest. Therefore, a direction of the rotation angle θ at which the detection signal 302 is obtained is the advancing direction.

As such, by using the measuring laser beam R1 having the absorption spectrum of the blood B, a guide of the advancing direction can be obtained even when whether or not the occluded area C is present near the photoacoustic guide wire 10 is not clear. In addition, by using the measuring laser beam R1 having the absorption spectrum of a biological body, in particular, the blood B having strong light absorption, the advancing direction can be determined with high accuracy.

When the advancing direction is determined, the rotation processing portion 116 operates the photoacoustic guide wire 10a to face the advancing direction. Next, the crushing laser beam R2 is emitted.

When the peaks of the detection signals obtained at individual rotation angles are the same, the signal processing portion 114 determines that the photoacoustic guide wire 10a can advance forward.

According to the second embodiment, the advancing direction of the photoacoustic guide wire 10a can be determined more easily than in the first embodiment.

### Third Embodiment

Next, the third embodiment of the present invention will be described with reference to FIG. 16.

In the third embodiment, unlike the first and second embodiments, the photoacoustic wave detection element 2 is not provided in the photoacoustic guide wire 10 (refer to FIG. 2).

### System

FIG. 16 is a schematic diagram illustrating a system structure of a photoacoustic guide support system Zb according to the third embodiment. In order not to make the drawings complicated, the occluded area is not illustrated in FIG. 16.

In the photoacoustic guide support system Zb illustrated in FIG. 16, unlike the first and second embodiments, a separate catheter 30 from the photoacoustic guide wire 10b is provided.

The catheter 30 has a tubular configuration, in which the photoacoustic guide wire 10b is movable (capable of advancing and retreating in an axial direction of the catheter 30).

In addition, in the example of FIG. 16, a photoacoustic wave detection element 2b is provided in a ring shape at the tip of the catheter 30.

An operation of the photoacoustic guide support system Zb according to the third embodiment is the same as the photoacoustic guide wire 10b according to the first embodiment, and thus the description thereof will not be repeated.

In addition, the photoacoustic guide wire 10b according to the third embodiment may have a configuration in which the measuring laser beam R1 and the crushing laser beam R2 are emitted in an oblique direction with respect to the central axis of the photoacoustic guide wire 10b and the rotation control device 20 is provided. That is, the photoacoustic guide support system Zb according to the third embodiment may have the same configuration as that of the second embodiment, except for the photoacoustic wave detection element 2b.

When the photoacoustic wave detection element 2 is provided on the side surface of the photoacoustic guide wire 10 or 10a as in the first and second embodiments, the thickness of the photoacoustic guide wire 10 or 10a increases by the thickness of the photoacoustic wave detection element 2. By providing the photoacoustic wave detection element 2b in the separate catheter 30 provided separately from the photoacoustic guide wire 10 as in the third embodiment, the diameter of the photoacoustic guide wire 10b that enters the occluded area C can be reduced. In particular, by providing the photoacoustic wave detection element 2b at the tip portion of the catheter 30, the photoacoustic wave detection element 2b can be provided to face a direction in which the photoacoustic wave W is incident. As a result, the detection ability of the photoacoustic wave W can be improved.

When the use of the photoacoustic guide wire 10b ends, the photoacoustic guide wire 10b is accommodated in the catheter 30.

When the photoacoustic wave W propagates not only due to the tissue of the occluded area C but also due to the blood B flowing into the crushed occluded area C. Accordingly, the photoacoustic guide wire 10b enters the tissue of the occluded area C, and even when the catheter 30 is present outside the occluded area C, the photoacoustic wave detection element 2b can receive the photoacoustic wave W.

The present invention is not limited to the embodiments and includes various modification examples. For example, the embodiments have been described in detail in order to easily describe the present invention, and the present invention is not necessarily to include all the configurations described above. In addition, a part of the configuration of one embodiment can be replaced with the configuration of another embodiment. Further, the configuration of one embodiment can be added to the configuration of another embodiment. Also, addition, deletion, and replacement of another configuration can be made for a part of the configuration each of the embodiments.

In addition, some or all of the above-described configurations, functions, portions 111 to 116 and 111a, the storage device 130, and the like may be realized by hardware, for example, by designing an integrated circuit. Also, as illustrated in FIGS. 3 and 11, the configurations, functions, and the like may be realized by software by a processor such as a CPU interpreting and executing a program that realizes each of the functions. Information such as a program, a table, or a file that realizes the functions can be stored not only in a hard disk (HD) but also in a storage device such as the memory 110 or a solid state drive (SSD) or in a storage medium such as an integrated circuit (IC) card, a secure digital (SD) card, or a digital versatile disc (DVD).

In addition, in each of the embodiments, the drawings illustrate control lines and information lines as considered necessary for explanations but do not illustrate all control lines or information lines in the products. It can be considered that almost of all components are actually interconnected.

What is claimed is:

1. A photoacoustic guide support system comprising:
a measuring laser beam generator that generates a measuring laser beam;
a guide wire that includes an optical fiber for emitting the measuring laser beam to a target, and a detection portion for detecting a photoacoustic wave generated when the target is irradiated with the emitted measuring laser beam; and
a signal processing portion that determines whether or not the guide wire is capable of advancing based on a detection signal obtained when the detection portion detects the photoacoustic wave, wherein
when the detection signal shows a predetermined pattern, the signal processing portion determines that the guide wire is capable of advancing forward and outputs the determination result to an output portion.

2. The photoacoustic guide support system according to claim 1, wherein
the measuring laser beam has an absorption spectrum corresponding to blood, and
when a signal having a predetermined value or higher is detected in the detection signal obtained from a predetermined distance or longer, the signal processing portion determines that the guide wire is capable of advancing forward.

3. The photoacoustic guide support system according to claim 1, wherein
the measuring laser beam has an absorption spectrum corresponding to a tissue of an occluded area of a blood vessel, and
when a signal having a predetermined value or higher is detected in the detection signal, the signal processing portion determines that the guide wire is capable of advancing forward.

4. The photoacoustic guide support system according to claim 1, wherein
a refraction portion that emits the measuring laser beam at a tilted angle with respect to a central axial direction of the optical fiber is provided in the optical fiber,
the optical fiber includes
a rotation controller that rotates the optical fiber, and
a rotation angle detection portion that detects a rotation angle of the optical fiber, and
the signal processing portion determines a rotation angle at which a signal showing a predetermined pattern is obtained among respective rotation angles as a direction in which the guide wire is to advance.

5. The photoacoustic guide support system according to claim 1, wherein
the measuring laser beam has an absorption spectrum corresponding to blood, and
when a signal having a predetermined value or higher is detected in the detection signal obtained from a predetermined distance or longer, the signal processing portion determines that the guide wire is capable of advancing to a rotation angle at which the detection signal is obtained.

6. The photoacoustic guide support system according to claim 1, wherein
the measuring laser beam has an absorption spectrum corresponding to a tissue of an occluded area of a blood vessel, and
when a signal having a predetermined value or higher is detected in the detection signal, the signal processing portion determines that the guide wire is capable of advancing to a rotation angle at which the detection signal is obtained.

7. The photoacoustic guide support system according to claim 1, wherein
a laser beam having a higher intensity than the measuring laser beam is emitted in an advancing direction of the optical fiber.

8. The photoacoustic guide support system according to claim 1, wherein
the detection portion is provided in the guide wire.

9. The photoacoustic guide support system according to claim 1, wherein
the detection portion is provided in a region other than the guide wire where the optical fiber is provided.

10. The photoacoustic guide support system according to claim 9, wherein
the detection portion is provided in a catheter where the guide wire is provided.

11. The photoacoustic guide support system according to claim 10, wherein
the detection portion is provided at a tip of the catheter.

12. A photoacoustic guide support method using a photoacoustic guide support system that includes
a measuring laser beam generator that generates a measuring laser beam,
a guide wire that includes an optical fiber for emitting the measuring laser beam to a target, and a detection portion detecting a photoacoustic wave generated when the target is irradiated with the emitted measuring laser beam, and
a signal processing portion that determines whether or not the guide wire is capable of advancing based on a detection signal obtained when the detection portion detects the photoacoustic wave,
the method comprising:
allowing the signal processing portion to determine that the guide wire is capable of advancing forward and to output the determination result to an output portion when a detection signal obtained by the detection portion detecting the photoacoustic wave shows a predetermined pattern.
